(19) United States
(12) Patent Application Publication
(10) Pub. No.: US 2019/0017006 A1
(43) Pub. Date: Jan. 17, 2019

(54) METHOD AND DEVICE FOR KEEPING SHAPE OF FRAGILE MATERIAL IN LIQUID

(71) Applicant: TERUMO KABUSHIKI KAISHA, Tokyo (JP)

(72) Inventors: Ryohei TAKEUCHI, Kanagawa (JP); Minori KIKUCHI, Kanagawa (JP); Eri NOGUCHI, Kanagawa (JP); Kouichirou YORI, Kanagawa (JP)

(73) Assignee: TERUMO KABUSHIKI KAISHA, Tokyo (JP)

(21) Appl. No.: 16/136,608

(22) Filed: Sep. 20, 2018

Related U.S. Application Data
(63) Continuation of application No. PCT/JP2017/011628, filed on Mar. 23, 2017.

(30) Foreign Application Priority Data
Mar. 28, 2016 (JP) 2016-063992

(51) Int. Cl. C12M 3/04; C12M 1/22; C12M 3/00; C12N 5/077

(52) U.S. Cl. CPC C12M 3/04; C12M 23/10; C12M 21/08; C12N 2533/30; C12M 1/22; C12N 2533/54; C12N 5/0658

(57) ABSTRACT

A method, a device, and an annular member are disclosed for keeping a shape of a fragile material in a liquid in a container. The method includes covering a surface of the liquid with a sealing material configured to melt and solidify according to a temperature change; and solidifying the sealing material to cover the surface of the liquid. The device includes a container configured to hold the fragile material; and a sealing material configured to melt and solidify according to a temperature change. The annular member includes an upper annular groove configured to receive a sealing material, the sealing material configured to melt and solidify according to a temperature change, the upper annular groove having an inner wall and an outer wall, and wherein a height of the inner wall is lower than a height of the outer wall.

### CROSS-REFERENCES TO RELATED APPLICATIONS

This application is a continuation of International Application No. PCT/JP2017/011628 filed on Mar. 23, 2017, which claims priority to Japanese Patent Application No. 2016-063992 filed on Mar. 28, 2016, the entire contents of both of which is incorporated herein by reference.

### TECHNICAL FIELD

The present disclosure generally relates to a method that retains a shape (or form) of a fragile object or fragile material in a liquid, and to a device and components of the device to put the method into practice.

### BACKGROUND ART

The recent methods for the treatment of heart disease are not designed to cope with severe cardiac failure. A common treatment for cardiac failure is by internal therapy that relies on a β-blocker or an angiotensin converting enzyme (ACE) inhibitor. For severe cardiac failure, however, the internal therapy is replaced by substitutional therapy, or surgical therapy, such as heart transplantation or use of an auxiliary artificial heart.

The severe cardiac failure that needs the surgical therapy mentioned above can result from various causes, such as advanced valvular disease, hyper myocardial ischemia, acute cardiac infarction and complications of the acute cardiac infarction, acute myocarditis, and chronic cardiac failure and its acute animus due to ischemic cardiomyopathy (ICM) and dilated cardiomyopathy (DCM).

The cause and severity of disease determine the selection of therapeutic regimen, such as valve formation or replacement, coronary artery by-pass, left ventricular formation, and mechanical auxiliary circulation.

It has been believed that the above-mentioned cardiac failure which results from an extreme decrease in the left ventricular function due to ICM or DCM can be treated effectively only by the substitutional therapy based on heart transplantation or artificial heart. Unfortunately, the substitutional therapy for patients of serious cardiac failure is not necessarily universally useful because of the chronic donor shortage, the necessity for continuous immune suppression, and the occurrence of complication.

There has recently arisen a new idea that problems with the therapy for serious cardiac failure should be tackled by regenerative medicine.

For example, serious cardiac infarction can lead to cardiac failure through the dysfunction of cardiac muscle cells, the proliferation of fibroblasts, and the fibrosing of interstitial tissues. The cardiac failure advances to damage cardiac muscle cells, giving rise to apoptosis. The cardiac muscle cells in this state rarely undergo cell division, which leads to a decrease in the number of cardiac muscle cells and a more decreased cardiac function.

It is considered that the patient of serious cardiac failure will have his/her cardiac function recovered effectively by cell transplantation. In fact, this new method is being put into clinical use with the help of autoskeletal muscle cells.

One of such examples has recently been disclosed, which is concerned with a cell culture and a method for production of the cell culture (JP 2007-528755A). According to this disclosure, the cell culture has a three-dimensional structure suitable for application to a heart and is composed of cells derived from any other part of the body than the cardiac muscle. The cell culture is obtained with the help of temperature responsive culture dishes devised by the tissue engineering.

The sheet-shaped cell culture mentioned above needs to be kept in a liquid held in a container, and the container needs to be transferred to the place where the sheet-shaped cell culture is used later. For stable transportation of the sheet-shaped structured object, a variety of methods and tools have been developed (JP 2003-070460A and JP 4749155B2).

Because of its inherently poor physical strength, the sheet-shaped cell culture mentioned above can be vulnerable (or susceptible) to wrinkling and breakage which occur when the surface of the liquid experiences storage waves (i.e., the surface is not flat and air bubbles are generated). This gives rise to a need for any means for the sheet-shaped cell culture to retain its form in the storage liquid. There has been developed a method for preventing the liquid surface from waving (JP 2013-192467A). This method includes incorporating the storage liquid for the sheet-shaped structured object with another liquid that forms a liquid-liquid interface between them in the container closed with a lid.

### SUMMARY OF DISCLOSURE

Under the circumstances mentioned above, the present inventors attempted to develop a method and device to allow a fragile object, such as sheet-shaped structured object, to retain its form in a liquid. They found that the container holding the liquid cannot be completely closed with the lid, because of air remaining between the lid and the liquid surface. Such remaining air results in the waving of the liquid. Moreover, in the case where the liquid containing the sheet-shaped structured object is covered with another liquid, the fragile sheet-shaped structured object fluctuates as the second liquid waves. In order to tackle the foregoing problems, a method, a device, and components of the method and the device are disclosed, which are intended for a fragile object to retain its form in a liquid.

In accordance with an exemplary embodiment, the liquid surface can be covered with a solid material so that the liquid containing the fragile object can escape (or avoid) movement. For example, the liquid surface can be covered with a solid more adequately if the liquid surface is covered with a sealing medium in a molten state and subsequently the molten sealing medium is solidified.

Thus, the method and device for allowing a fragile object to retain its form in a liquid held in a container as disclosed here may involve the following aspects [1] to [7].

[1] A method for allowing a fragile object to retain its form in a liquid held in a container, the method including: covering the surface of the liquid with a molten sealing medium that melts and solidifies according to temperature change; and solidifying the sealing medium covering the liquid surface.

[2] The method as defined in paragraph [1] above, in which the fragile object is a sheet-shaped cell culture of skeletal myoblasts.

[3] The method as defined in paragraph [1] or [2], in which the sealing medium has a melting point of 0° C. to 45° C. and a solidifying point of 0° C. to 45° C.

[4] The method as defined in any one of paragraphs [1] to [3], in which the sealing medium includes at least one selected from the group consisting of fatty ester, gelatin, polysaccharide, and polymeric compound.

[5] A device that allows a fragile object to retain its form in a liquid, the device including: a container to hold the fragile object in the container; a sealing medium that melts and solidifies according to temperature change; and an optional lid for the container, with the sealing medium in a solidified state adhering to the container and/or the lid.

[6] The device as defined in paragraph [5], in which the container holding the fragile object has a groove to fix the sealing medium, with the groove having an inner wall and an outer wall such that the former is lower in height than the latter.

[7] An annular member capable of adhering to an edge of a container holding a fragile object in the container, the annular member having an annular groove to support a sealing medium in the annular groove that melts and solidifies according to temperature change, with the annular groove having an inner wall and an outer wall such that the former is lower in height than the latter.

In accordance with an exemplary embodiment, the present disclosure produces an effect of efficiently covering the liquid surface owing to the sealing medium that melts and solidifies in response of temperature change. Moreover, it produces an effect of efficiently preventing the liquid from moving owing to the sealing medium that solidifies on the liquid surface. It also produces an effect of protecting the fragile object in the liquid from contamination because the liquid is isolated from the outside air.

In accordance with an exemplary embodiment, the present disclosure is particularly effective in preventing the fragile object from wrinkling, breaking, and deforming when its container is moved, thereby keeping it physically stable.

Moreover, the present disclosure provides a method, a device, and components of the method and the device, which are relatively inexpensive and simple, for the fragile object to retain its form in the liquid.

### BRIEF DESCRIPTION OF THE DRAWINGS

FIG. 1 is a sectional perspective view depicting a device pertaining to a first exemplary embodiment of the present disclosure.

FIG. 2 is a sectional perspective view depicting a device pertaining to a second exemplary embodiment of the present disclosure.

FIGS. 3A to 3C are schematic sectional views depicting modified examples of the device depicted in FIG. 2.

FIG. 4 is a sectional perspective view depicting a member pertaining to a third exemplary embodiment of the present disclosure.

FIGS. 5A to 5D are schematic sectional views depicting modified examples of the member depicted in FIG. 4.

FIG. 6 is a photograph showing a gelled gelatin which adheres to the inner side surface of a Petri dish.

FIG. 7 is a photograph showing a gelled gelatin which adheres to the inner surface of a lid.

FIG. 8 is a photograph showing a gelled gelatin that covers water in a Petri dish.

### DETAILED DESCRIPTION

One aspect of the present disclosure covers a method for allowing a fragile object to retain its form in a liquid held in a container, the method including: covering the surface of the liquid with a molten sealing medium that melts and solidifies according to temperature change; and solidifying the sealing medium covering the liquid surface.

The term “fragile object” as used in this specification denotes any object which has such low physical strength as to deform or break when the liquid moves. Such objects, for example, can include objects having a thin-walled part, objects having a belt-shaped form, and objects having a sheet-shaped form. Examples of the sheet-shaped object unrestrictedly include sheet-shaped structured objects, for example, filmy tissue formed on a flat film from any material derived from a living organism such as sheet-shaped cell culture, and various kinds of film formed from plastic, paper, woven cloth, unwoven cloth, metal, polymer, and/or lipid. Preferable among the fragile objects are objects that do not decompose or disintegrate in a liquid. The sheet-shaped structured object may vary in shape (for example, polygon, circle, or the like), diameter, width, thickness, etc. Moreover, the sheet-shaped structured object in the present disclosure may be of monolayer or multilayer. The multilayered object may have layers joined together or separated from one another. The term “joined” does not necessarily mean that overlapped parts (i.e., parts that extend over and cover) of the layers are entirely joined to one another.

The term “sheet-shaped cell culture” as used in this specification denotes a cell culture which resembles a sheet having cells joined together. The sheet-shaped cell culture typically consists of one layer of cells, but the sheet-shaped cell culture may consist of two or more layers of cells. The cells may join together directly or indirectly with a substance existing among the cells. Such an intervening substance unrestrictedly includes any substance that is able to join cells together at least physically (mechanically); an example is an extracellular matrix. The intervening substance should preferably be a substance, which is derived from cells, especially, for example, those cells that constitute the cell culture. The cells of the cell culture can be joined together at least physically (mechanically) and may be further joined together functionally, for example chemically or electrically.

The sheet-shaped cell culture involved in the present specification is comprised of any cells capable of forming the above-mentioned structure. Examples of such cells unrestrictedly include adherent cells. The adherent cells can include adherent somatic cells and stem cells. The somatic cells can include cardiac muscle cells, fibroblasts, epithelial cells, endothelial cells, hepatic cells, pancreatic cells, nephrocytes, adrenal cells, periodontal membrane cells, gingival cells, periosteal cells, dermal cells, synovial cells, and cartilage cells. The stem cells can include myoblasts, tissue stem cells such as cardiac stem cells, embryonic stem cells, pluripotent stem cells such as iPS (induced pluripotent stem) cells, and mesenchymal stem cells. The somatic cells may be those that are obtained by differentiation from stem cells, especially iPS cells. The cells capable of forming the sheet-shaped cell culture unrestrictedly include myoblasts (for example, skeletal myoblasts), mesenchymal stem cells (for example, those which are derived from marrow, fat tissues, peripheral blood, skin, hair root, muscle tissue, endometrium, placenta, and cord blood), cardiac muscle cells, fibroblasts, cardiac stem cells, embryonic stem cells, iPS cells, synovial cells, chondrocytes, epithelial cells (for example, oral mucosa epitheliocytes, retinal pigment epitheliocytes, and nasal mucosa epitheliocytes), endothelial cells (for example, blood endothelial cells), hepatocytes (for example, hepatic mesenchymal cells), pancreatic cells (for example, islet cells), nephrocytes, adrenal cells, periodontal membrane cells, gingival cells, periosteal cells, and dermal cells. In the present specification, the preferable cells can be myoblasts, especially skeletal myoblasts, which form monolayered cell cultures.

In accordance with an exemplary embodiment, cells derived from any living organism that can be cured with the help of cell culture can be used. Such living organisms unrestrictedly include human, primate, dog, cat, pig, horse, goat, sheep, etc. The sheet-shaped cell culture may be prepared from one species of cells or more than one species of cells. According to a preferred embodiment of the present disclosure, the cell culture should be prepared from more than one species of cells in such a way that the major cells account for equal to or more than 65%, preferably equal to or more than 70%, and more preferably equal to or more than 75%, when the cell culture is prepared completely, in the case of skeletal myoblasts, for example.

The sheet-shaped cell culture used in the present disclosure may be a cultured tissue in a sheet form which is obtained by inoculating cells onto a scaffold (for cell culturing) and culturing; however, the sheet-shaped cell culture should preferably be a sheet-shaped cell culture, which is composed solely of substances derived from the cells constituting the cell culture.

In accordance with an exemplary embodiment, the sheet-shaped cell culture may be prepared by known methods.

According to one aspect of the present disclosure, the sheet-shaped cell culture is a sheet-shaped cell culture of skeletal myoblasts. The sheet-shaped cell culture of skeletal myoblasts can be vulnerable to breaking by its own weight even when a part of the sheet-shaped cell culture is pinched. Thus, the sheet-shaped cell culture cannot be transported in its isolated form, or the sheet-shaped cell culture can present extreme difficulties in restoring the original form of the sheet-shaped cell culture once it is folded. Consequently, keeping the sheet form in a liquid can be of great significance.

In the present specification, the liquid in the container is composed of at least one component, which is unrestrictedly exemplified by such liquid as water, aqueous solution, non-aqueous solution, suspension, emulsion, and the like.

Moreover, in the present specification, the liquid may contain solids (derived from the scaffold), bubbles, and any other non-liquid components as long as the liquid is a fluid having fluidity as a whole.

In accordance with an exemplary embodiment, the liquid in the container may be composed of any components that do not affect the fragile object. In the case where the fragile object is a film composed of materials derived from a living organism, the components constituting the liquid in the container should preferably be ones, which are compatible with a living organism from the standpoint of biological stability and long-term storage stability. Biological stability means the complete or substantial absence of undesirable reactions such as inflammatory, immunity, and intoxication to the living organism and cells. Examples of such components include water, saline, physiological buffer solution (such as HBSS, PBS, EBSS, Hepes, and sodium bicarbonate), culture medium (such as DMEM, MEM, F12, DMEM/F12, DME, RPMI1640, MCDB, L15, SkBM, RITC80-7, and IMDM), sugar solution (such as sucrose solution and Ficoll-paque® PLUS), sea water, serum-containing solution, Renografin® solution, metrizamide solution, meglumine solution, glycerin, ethylene glycol, ammonia, benzene, toluene, acetone, ethyl alcohol, oil, mineral oil, animal oil, vegetable oil, olive oil, colloidal solution, liquid paraffin, turpentine oil, linseed oil, and castor oil.

In accordance with an exemplary embodiment, in the case where the fragile object is a sheet-shaped cell culture, the liquid in the container should preferably be composed of components that can help ensure stable cell storage, contain minimal oxygen and nutrients essential for cell existence, and have an osmotic pressure low enough not to break cells. Examples of the components to meet these requirements unrestrictedly can include saline, physiological buffer solution (such as HBSS, PBS, EBSS, Hepes, and sodium bicarbonate), culture medium (such as DMEM, MEM, F12, DMEM/F12, DME, RPMI1640, MCDB, L15, SkBM, RITC80-7, and IMDM), and sugar solution (such as sucrose solution and Ficoll-paque® PLUS).

The liquid in the container is not specifically restricted in amount, for example, a desirable amount is such that the solution level is high enough to prevent the fragile object from moving. For example, in the case where the sheet-shaped cell culture of skeletal myoblasts has a diameter of approximately 10 mm or 20 mm, the height of the liquid level should be, for example, 0.1 mm to 11 mm or 0.1 mm to 16 mm, respectively. In addition, the amount of the liquid should preferably be, for example, 1 mL to 100 mL from the standpoint of cooling the sealing medium (sealing medium) that covers the liquid surface. The liquid should preferably be a liquid that has a specific gravity smaller than that of the fragile object, so that the fragile object will not float on the liquid surface to come into contact with the sealing medium. Moreover, the specific gravity of the liquid should preferably be equal to that of the fragile object, so that the fragile object stably keeps its form without moving in the liquid when the container inclines.

In this specification, the term “temperature change” denotes the change in temperature which the sealing medium experiences upon heating and cooling. The term “melt” denotes the phenomenon that the sealing medium partly or entirely melts upon heating. The term “solidify” denotes the phenomenon that the sealing medium in a molten state partly or entirely solidifies or semisolidifies (for example, coagulates). In accordance with an exemplary embodiment, in the case where the sealing medium is composed of those components which undergo sol-gel transformation due to temperature change, the term “melt” denotes the phenomenon that the sealing medium dissolves in gel and the term “solidify” denotes the phenomenon that the sealing medium becomes gel. Therefore, in this specification (or disclosure), the term “melting point” can be synonymous with “gel dissolving temperature” and the term “solidifying point” can be synonymous with “gelling temperature.”

In this specification (or disclosure), the sealing medium can include a sealing medium which is composed of a single component or a sealing medium which is composed of two or more components. In accordance with an exemplary embodiment, the sealing medium can denote any material which melts upon heating to cover in a molten state the liquid surface, and which solidifies upon cooling to form a sealing lid on the liquid surface. In accordance with an exemplary embodiment, the sealing medium should preferably be composed of components stable to oxidation and hydrolysis. The sealing medium may be made stable to oxidation and hydrolysis by incorporation with known stabilizers in the field, such as antioxidant, weathering agent, UV absorber, and hydrolysis inhibitor. In this way, the sealing medium in contact with the liquid can be saved from oxidation and hydrolysis, and the sealing medium which has solidified on the liquid surface can isolate the liquid from the external air while keeping the properties of the sealing medium. This can help realize the long-term storage of the fragile object in the liquid.

In the case where the fragile object consists of cells or contains cells, the sealing medium should preferably be composed of components compatible with living organisms, which would cause no or little immune reaction, inflammatory reaction, or toxic reaction undesirable for living tissues and cells. In addition, the sealing medium should preferably be one which melts and solidifies in response to temperature change to produce no adverse effects on cells. In accordance with an exemplary embodiment, any sealing medium suitable for the transportation, storage and long-term storage of the fragile object may be prepared from various components which would be adequately selected by those who are skilled in the art from the standpoint of the range of temperature change and the compatibility to living organisms.

The sealing medium is not specifically restricted in melting point and solidifying point. In accordance with an exemplary embodiment, the melting point, for example, should be 0° C. to 45° C., preferably 10° C. to 30° C., and more preferably 15° C. to 25° C. The solidifying point, for example, should be 0° C. to 45° C., preferably 10° C. to 30° C., and more preferably 15° C. to 25° C. For the sealing medium to solidify at room temperature, it should preferably have a solidifying point, for example, ranging from 30° C. to 37° C. Moreover, the sealing medium should preferably have a melting point, for example, ranging from 0° C. to 37° C. so that it gives off a minimum of latent heat of fusion that could affect cells contained in the fragile object in the liquid.

In accordance with an exemplary embodiment, the sealing medium should be used in such an amount as to cover the entire liquid surface, with the thickness of the sealing medium being, for example, 1 mm to 20 mm, preferably 1 mm to 10 mm, more preferably 3 mm to 5 mm.

The sealing medium can include, for example, fatty acid ester (wax with a solidifying point of 35° C. to 50° C., animal fat with a solidifying point of 25° C. to 50° C., and vegetable fat), gelatin (with a gelling temperature of 15° C. to 30° C.), polysaccharide, and polymeric compound. The polysaccharide and polymeric compound can be exemplified by pectin (25° C. to 45° C.), agar-agar (25° C. to 45° C.), carageenan (25° C. to 60° C.), cellulose (30° C. to 40° C.), gellan gum (30° C. to 40° C.), methyl cellulose (30° C. to 55° C.), xanthan gum+locust bean gum (35° C. to 60° C.), and curdlan (30° C. to 60° C.), with the respective gelling temperatures of the sealing medium indicated in parentheses. The polymeric compound can include telechelic PEG and polyethylene, which undergo sol-gel conversion owing to the hydrophobic mutual reaction. The sealing medium in the present disclosure may be used alone or in combination with one another. An adequate sealing medium will be selected by those who are skilled in the art.

According to the present disclosure, the sealing medium is not specifically restricted in specific gravity so long as the sealing medium can be placed on the liquid holding the fragile object. In accordance with an exemplary embodiment, the preferred specific gravity is smaller than that of the liquid in the container and that of the fragile object.

In the present specification, the sealing medium may be melted in any manner without specific restrictions, for example, melting may be accomplished by direct heating or indirect heating. For example, the sealing medium may be melted by exposing the sealing medium to hot air or by heating the container to which the sealing medium is fixed.

In the present specification, there are no specific restrictions on the process of covering the liquid surface with the sealing medium in a molten state. A preferable process for the fragile object such as sheet-shaped cell culture of skeletal myoblasts consists of slowly dropping the sealing medium from a pipette. This process can help prevent the fragile object from being damaged. Another possible process consists of dropping the sealing medium along the inner side surface of the container. The process of dropping the sealing medium along the inner side surface of the container allows the sealing medium to be dropped slowly to the liquid surface. The sealing medium may cover the liquid surface entirely or partly.

In the present specification, there are no specific restrictions on the process of solidifying the sealing medium covering the liquid surface. The sealing medium may be solidified by direct cooling or indirect cooling in which case the container is cooled. The cooling may be accomplished by means of a cooling device or a liquid placed in the container, or by natural cooling with the container standing at room temperature. The solidified sealing medium covering the liquid surface can help prevent the liquid surface from shaking because the periphery of the sealing medium in the radial direction can be fixed to the inner side surface of the container.

The foregoing procedure by which the liquid in the container holding the fragile object is covered with the sealing medium in a molten state, which is subsequently solidified, can help prevent the fragile object in the liquid from disintegration when the container is moved. That is, the liquid that is incompressible does not move and hence does not apply any force to deform the fragile object so long as there is no gas between the liquid surface and the solidified sealing medium. In this case, the fragile object may float in the liquid in the container or may exist on the bottom of the container. The sealing medium in a molten state can properly cover the liquid surface even when the liquid height varies depending on the container.

The method according to one aspect of the present disclosure may include an additional step of removing the sealing medium after the sealing medium has solidified. With the sealing medium removed, the fragile object can be taken out (or removed) from the liquid when the fragile object is used. The step of removing the sealing medium may include a step of melting the sealing medium. The procedure of melting the sealing medium allows the sealing medium to be removed relatively easily. Further, the step of removing the sealing medium may include a step of melting the sealing medium and then adding the liquid to the container. The procedure of melting the sealing medium and then adding the liquid to the container increases the volume of the liquid in the container and causes the molten sealing medium to be pushed out of the container. Thus, the sealing medium can be removed relatively easily.

In one aspect of the present disclosure, the liquid used to form the fragile object can be removed and replaced by another liquid that supports the fragile object to be preserved. The procedure of replacing the liquid with another liquid can help eliminate the necessity of mechanically taking out the fragile object by means of hands or tools, for transfer to another container. This procedure can be useful particularly for fragile sheet-shaped cell cultures. The necessary steps to achieve the purpose with sheet-shaped cell cultures can include preparing a sheet-shaped cell culture in a culture vessel filled with a culture medium (solution), removing the culture medium, filling the culture vessel with a preserving liquid, and pouring the sealing medium into the culture vessel. The sheet-shaped cell culture that experiences these steps can avoid unnecessary contact with anything other than the liquid during a period from preparation to transfer, and to transplantation. In accordance with an exemplary embodiment, the methods and devices disclosed can help limit chances for damage and contamination to the sheet-shaped cell culture.

A further aspect of the present disclosure covers a device that allows a fragile object to retain the form of the fragile object in a liquid. The device can include: a container to hold the fragile object in the container; a sealing medium that melts and solidifies in response to temperature change; and an optional lid for the container, with the sealing medium in a solidified state adhering to the container and/or the lid.

In the present specification, the container is not specifically restricted so long as the container is capable of holding the fragile object, the liquid, and the sealing medium in the container and is capable of preventing the liquid from leaking form the container. Any container, including commercial ones, may be used. For example, the container may be a container, which is made of such material as polyethylene, polypropylene, Teflon (registered trademark), polyethylene terephthalate, polymethyl methacrylate, nylon 6,6, polyvinyl alcohol, cellulose, silicone, polystyrene, glass, polyacrylamide, polydimethylacrylamide, and metal (for example, iron, stainless steel, aluminum, copper, and brass). In accordance with an exemplary embodiment, the container should preferably have at least one flat bottom (i.e., a portion of the bottom of the container can be flat and another portion of the bottom of the container can be angled or rounded) that can help maintain the shape of the fragile object. For example, the container can be a Petri dish, cell culture dish, and cell culture bottle.

In the present specification, the container may have a lid which is placed on the container to cover the upper part of the container, thereby achieving tight sealing (i.e., such that liquid does not leak from the container). Any lid, including commercial lids, may be used. In addition, the lid may be a discoid drop lid (disc shaped lid) which has a smaller diameter than the inside diameter of the container so that the discoid drop lid can be placed on the liquid surface. The lid may be one which is made of such material as polyethylene, polypropylene, Teflon®), polyethylene terephthalate, polymethyl methacrylate, nylon 6,6, polyvinyl alcohol, cellulose, silicone, polystyrene, glass, polyacrylamide, polydimethylacrylamide, and metal (for example, iron, stainless steel, aluminum, copper, and brass).

The device according to the present disclosure may include a container, a sealing medium, and a lid (optional). The container may or may not hold the fragile object and the liquid in the container (i.e., the container can be provided before the fragile object and/or the liquid is placed in the container). The solidified sealing medium may be fixed to the container, or to the lid, or to both the container and lid. The device according to the present disclosure may be used in such a way that the container holds the liquid and the fragile object in the container, the sealing medium is fixed to the container and/or the lid is melted so that that sealing medium covers the liquid surface, and the sealing medium covering the liquid surface is solidified. This procedure permits the liquid surface to be covered with the lid. Moreover, this procedure prevents air from entering the gap between the liquid surface and the lid. The result is that even when the container is shaken during transportation, the liquid in the container remains stable without waving and moving, the fragile object remains stable, and the liquid avoids contact with outside air, and which allows the shape of the fragile object in the liquid to be retained without deformation and contributes to the long-term storage of the fragile object.

The preferable embodiments of the present disclosure will be described below with reference to the accompanying drawings.

### First Embodiment

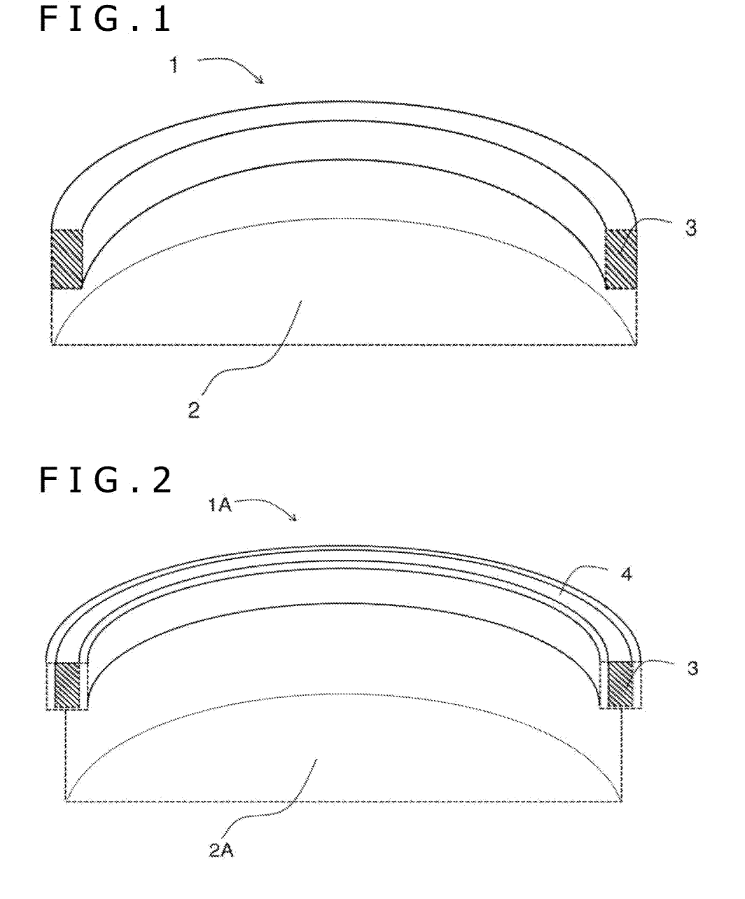

The following is a description of a device 1 pertaining to the first embodiment of the present disclosure, the device 1 being depicted in FIG. 1, which is a sectional perspective view. Incidentally, the drawings in the present application depict various components in an exaggerated scale, differing from actual dimensions, to facilitate explanation.

It is noted from FIG. 1 that the device 1 pertaining to the first embodiment of the present disclosure includes a container 2 and a sealing medium 3. The container 2 is a round Petri dish having an opening. The sealing medium 3 assumes an annular shape corresponding to the shape of the inner side surface of the container 2 and fixes on the upper part of the inner side surface of the container 2. When the device 1 is put to use, the container 2 is allowed to hold a liquid and a fragile object in the container, and then heated so that the sealing medium 3 fixed to the container 2 melts. The sealing medium 3 in a molten state covers the liquid surface, and subsequently, the molten sealing medium 3 covering the liquid surface is allowed to solidify so that the sealing medium 3 forms a lid, which covers the liquid surface.

According to the present embodiment, the sealing medium 3 adheres to the upper part of the container 2, so that the sealing medium 3 can cover the liquid held in the container 2 under the sealing medium 3 from above when the sealing medium 3 melts. Moreover, the sealing medium 3 adheres to the inner side surface of the container 2, so that the molten sealing medium 3 can slowly drip down along the inner side surface of the container 2. In addition, the sealing medium 3 assumes an annular shape, so that the sealing medium 3 can drip down uniformly in the circumferential direction of the container 2.

The foregoing embodiment, in which the sealing medium 3 adheres to the upper part of the container 2, is not intended for restrictions. For example, the sealing medium 3 may adhere to the bottom, or the lower part, of the container 2. The sealing medium 3 may take on any shape in conformity with the shape of the container 2. For example, the sealing medium 3 may assume a discoid shape (disk shaped) that entirely covers the opening of the container 2. The discoid sealing medium 3 can drip on the entire liquid surface from above when the discoid sealing medium 3 melts. In addition, the sealing medium 3 may adhere to the inner surface of the lid (not depicted) of the container 2 rather than adhering to the container 2. In this case, the sealing medium 3 in a molten state covers the inner surfaces of the lid and the container 2 and the liquid surface, so that the sealing medium 3 fixes the lid and the container 2 (i.e., the sealing medium 3 solidifies, which fixes or secures the lid on the container 2), thereby ensuring the tight sealing and the long-term storage of the fragile object. This can be facilitated if the inner surface of the lid is inclined so that the sealing medium 3 in a molten state is led to the inner side surface of the container 2. As mentioned above, the sealing medium 3 will function as desired regardless of whether the sealing medium 3 is fixed to the container 2, to the lid, or to both the container 2 and the lid.

As mentioned above, the device 1 according to the first embodiment of the present disclosure is characterized in that the surface of the liquid in the container is properly covered and air is absent between the liquid surface and the sealing medium. This produces the effects of protecting the fragile object from moving even when the container shakes during transportation and the liquid in the container waves, and can also prevent the liquid from coming into contact with external air. The foregoing effects permit the fragile object to be stored for a relatively long period of time without deformation in the liquid.

### Second Embodiment

The following is a description of a device 1A pertaining to the second embodiment of the present disclosure, the device 1A being depicted in FIG. 2, which is a sectional perspective view. Incidentally, the same components as those of the device 1 of the first embodiment are indicated by the identical symbols, with their explanation omitted.

As depicted in FIG. 2, the device 1A pertaining to the second embodiment of the present disclosure includes a container 2A and the sealing medium 3. The container 2A has an annular groove 4 at an opening edge of the container 2A. The annular groove 4 permits a lid (not depicted) of the container 2A to fit in and also holds the sealing medium 3 that adheres to the inside of container 2A. When the device 1A is put to use, the container 2A is filled with a liquid and a fragile object is placed in the liquid, and the container 2A is closed with the lid, which is placed on the groove 4 and heated subsequently. Upon heating, the sealing medium 3 melts to cause the lid to enter the inside of the groove 4 thereby pushing the sealing medium 3 out of the groove 4. The sealing medium 3, which has been pushed out, drips down along the inner side surface of the container 2A to cover the liquid surface. The next step is to solidify the sealing medium 3 on the liquid surface and in the groove 4. This step causes the sealing medium 3 to simultaneously cover the liquid surface and fix the lid to the container 2A, and which results in the container 2A becoming tightly sealed.

According to this embodiment, the groove 4 has an annular shape; but this is not necessarily essential. The shape of the groove 4 may vary in conformity with the shape of the lid. The groove 4 may be of annular shape, which makes the lid fit rather easily, in the case where the container 2A is a round Petri dish, with the opening and outer side surface of the container 2A covered by a round lid. The groove 4 may have any volume that is large enough to accommodate the sealing medium 3 that can fix the lid to the container 2A and cover the liquid surface.

As mentioned above, the device 1A pertaining to the second embodiment of the present disclosure is able to properly cover the surface of the liquid in the container and wherein air does not enter between the liquid surface and the sealing medium. This produces the effects of protecting the fragile object from moving even when the container shakes during transportation and the liquid in the container waves, and can also help prevent the liquid from coming into contact with external air. The foregoing effects permit the fragile object to be stored for a relatively long period of time without deformation in the liquid.

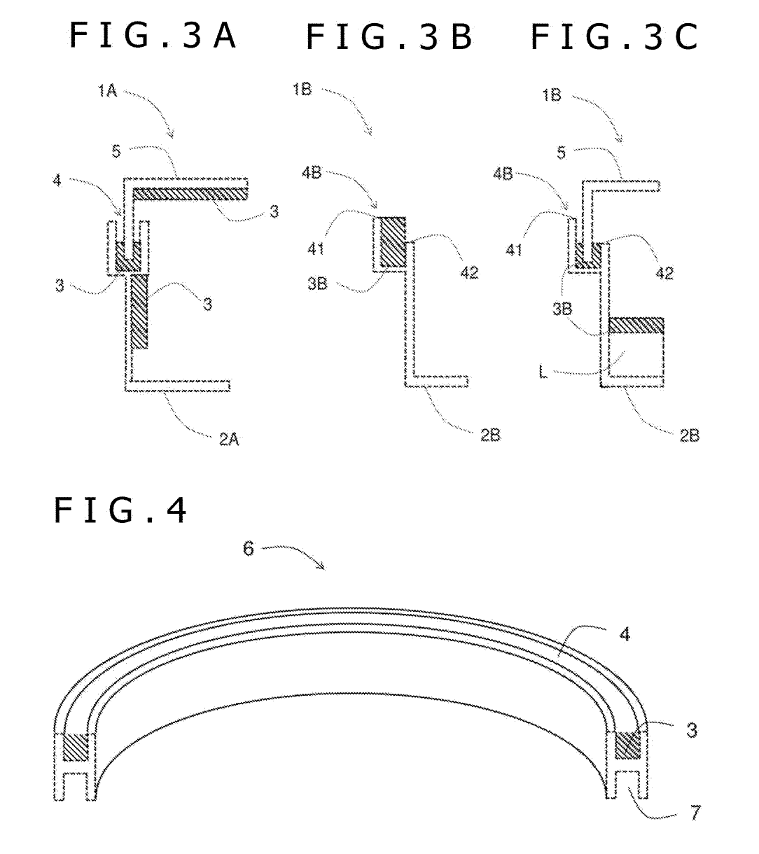

The device depicted in FIG. 2 may be modified as depicted in FIGS. 3A to 3C, which are schematic sectional views. Incidentally, the same components as those of the device of the second embodiment are indicated by the identical symbols, with their explanation omitted.

### FIRST MODIFIED EXAMPLE

As depicted in FIG. 3A, a device 1A pertaining to the first modified example of the present disclosure is characterized in that the sealing medium 3 adheres to the inside of the groove 4 of the container 2A, to the inner side surface of the container 2A, and to a lid 5. When the device 1A of the present disclosure is put to use, the container 2A is given a liquid and a fragile object and the lid 5 is placed in the groove 4 of the container 2A, and subsequently the resulting assembly of the container 2A, the liquid, the fragile object, and the lid 5 is heated so that the sealing medium 3 melts. The molten sealing medium 3, which is on the inner side surface of the container 2A, drips down to the liquid surface from the outer side in the radial direction of the container 2A. The molten sealing medium 3 adhering to the lid 5 drips to the liquid surface from the inner side in the radial direction of the container 2A, and the molten sealing medium 3 in the groove 4 of the container 2 enters the gap between the groove 4 and the lid 5. The result is that the entire liquid surface is covered rapidly and the lid 5 and the container 2A are fixed together.

According to this modified example, the sealing medium 3 adheres to the inner side surface of the container 2A, the groove 4, and the lid 5. This structure may be modified such that the sealing medium 3 adheres to at least one of the inner side surface of the container 2A, the groove 4, and the lid 5. For example, in the case where the sealing medium 3 adheres to only the lid 5, the desired object will be achieved if the amount of the sealing medium 3 is properly adjusted so that the molten sealing medium 3 fills the groove 4 of the container 2A along the inner surface of the lid 5 and the molten sealing medium 3 overflows the groove 4 so as to entirely cover the liquid surface. Therefore, the lid 5 of the container 2A may have the inner surface of lid 5 so formed as to incline toward the side surface of the container 2A. This permits the sealing medium 3 to flow toward the groove 4 and the inner side surface of the container 2A.

### SECOND MODIFIED EXAMPLE

As depicted in FIG. 3B, a device 1B pertaining to the second modified example of the present disclosure is characterized in that a container 2B has a groove 4B on the edge of the container 2B to which the lid 5 of the container 2B fits. In accordance with an exemplary embodiment, an inner wall 42 of the groove 4B is formed as to exist along the inner side surface of the container 2B. In addition, the groove 4B is formed such that the inner wall 42 of the groove 4B is lower than the outer wall 41 of the groove 4B. The device 1B of the present disclosure is used in such a way that a sealing medium 3B to be fixed to the inside of the groove 4B is higher than the inner wall 42 of the groove 4B. The container 2B is given a liquid L and a fragile object, and the assembly of the container 2B, the liquid L, and the fragile object is heated. The sealing medium 3B melts in the groove 4B, and the sealing medium 3B existing above the top of the inner wall 42 of the groove 4B drips down along the inner side surface of the container 2B to cover the surface of the liquid L (FIG. 3C). As the sealing medium 3B covering the liquid surface solidifies, the outer part in the radial direction of the sealing medium 3B adheres to the inner side surface of the container 2B, such that the liquid surface hardly moves (for example, the surface of the liquid does not generate air bubbles). In addition, a lower side of the sealing medium 3B is kept (or remains) in the groove 4B, thereby fixing the lid 5 to the inside of the groove 4B.

The modified example mentioned herein is characterized in that the groove 4B is constructed such that the inner wall 42 is lower than the outer wall 41. The groove 4B may be constructed in this way entirely or partly. The object of this structure may be achieved, for example, if the groove 4B has a hole made at a certain part of the groove 4B which causes the inside of the groove 4B to communicate with the inner side surface of the container 2B.

As mentioned above, the device 1B according to this modified example permits the molten sealing medium 3B to be introduced into the container 2B from the groove 4B even if the lid 5 is not fit into the groove 4B. Therefore, the device according to the present disclosure does not necessarily include the lid.

Moreover, in the case where the lid is a discoid drop lid (disc shaped lid) as mentioned above, the lid may be placed on the liquid surface, with the sealing medium fixed to at least one side of the lid, and then the sealing medium is melted and solidified. This produces an advantage that a side surface of the drop lid can be fixed to the inner side surface of the container by the sealing medium so that the liquid surface is covered with a solid and the amount of the sealing medium can be saved (or minimized). The drop lid may be made of a material that is harder than the solidified sealing medium. The resulting drop lid will hold the liquid surface more firmly so that the drop lid is sure to prevent the liquid from moving even when the container is inclined excessively.

The drop lid may be removed by breaking or melting the sealing medium. The procedure for removal of the drop lid may be made relatively easy if the drop lid is provided with a notch, opening, tab, or the like. For example, the opening may be an opening, which can be opened and closed, so that the opening is closed when the drop lid is fixed to the container for the long-term storage of the fragile object in the liquid and the opening is opened for air to pass so that the drop lid can be removed relatively easily without the necessity of melting the sealing medium.

The foregoing demonstrates that the devices 1A and 1B pertaining to the modified examples of the present disclosure adequately cover the surface of the liquid in the container and prevent air from entering the gap between the liquid surface and the sealing medium. This produces the effect of protecting the liquid in the container from waving and exposing the liquid to external air and hence protecting the fragile object from moving even when the container experiences vibration during transportation. The effect mentioned above contributes to storing the fragile object stably for a relatively long period of time, with the form of the fragile object being maintained.

Another aspect of the present disclosure covers an annular member capable of adhering to an edge of a container holding in the container a fragile object, the annular member having an annular groove to support in the annular member, a sealing medium that melts and solidifies according to temperature change, with the annular groove having an inner wall and an outer wall such that the inner wall is lower in height than the outer wall.

### Third Embodiment

The following is a description of a member pertaining to the third embodiment of the present disclosure. FIG. 4, which is a sectional perspective view, depicts a member pertaining to the third embodiment of the present disclosure. Incidentally, the same components as those of the devices in the first and second embodiments are indicated by the identical symbols, with their explanation omitted.

It is to be noted from FIG. 4 that a member 6 pertaining to the third embodiment of the present disclosure is an annular member that can adhere to the edge of a container (not depicted) to hold a fragile object in the container. The member 6 has the groove 4 to which a lid (not depicted) of the container fits, and the groove 4 holds and adheres the sealing medium 3 in the groove 4. In addition, the member 6 has a fitting part 7 formed in the member 6, and which can be firmly fixed on the edge of the container. According to this embodiment, the fitting part 7 and the groove 4 can have an annular shape (i.e., ring-shape).

The member 6 is used in such a way that the fitting part 7 of the member 6 can be fitted to the edge of the container holding in the container a liquid and the fragile object, the member 6 can be firmly fixed to the container, and the lid can be placed on the groove 4 of the member 6. Subsequently, the entire system of the container, the liquid, the member 6, and the lid is heated so that the sealing medium 3 melts and the lid enters the groove 4 to push out the sealing medium 3 to cover the liquid surface along the inner side surface of the member 6. The sealing medium 3 covering the liquid surface is cooled to solidify by the liquid in the container, with the result that the lid adheres to the container and the sealing medium 3 tightly closes the liquid in the container. In accordance with an exemplary embodiment, the sealing medium 3 drips down onto the liquid surface along the inner side surface of the member 6 such that the sealing medium 3 hardly adheres to the container 2 because of the sealing medium 3 dripping down onto the liquid surface. It is only necessary to take away the member 6 from the container in order to remove easily the sealing medium 3 from the liquid surface. It is also possible to remove the sealing medium 3 relatively easily if a hole for air to pass through is made in the solidified sealing medium 3. Such a hole for air to pass through may be made in the member 6 so as to open and close so that the sealing medium 3 can be removed relatively easily without the necessity for making a hole in the member 6. In this manner, the member 6 can be freely attached and detached to and from any commercial container and lid and permits the solidified sealing medium 3 to be removed relatively easily from the liquid surface.

The member 6 may be one which is made of such material as polyethylene, polypropylene, Teflon (registered trademark), polyethylene terephthalate, polymethyl methacrylate, nylon 6,6, polyvinyl alcohol, cellulose, silicone, polystyrene, glass, polyacrylamide, polydimethylacrylamide, and metal (for example, iron, stainless steel, aluminum, copper, and brass). The member 6 should preferably be flexible from the standpoint of that the member can be relatively easy attached and detached from the container.

It has been demonstrated above that the member 6 pertaining to the third embodiment of the present disclosure properly covers the surface of the liquid in the container, thereby preventing air from entering the gap between the liquid surface and the sealing medium, with the result that the liquid in the container does not wave nor does the liquid come into contact with external air and the fragile object in the liquid does not move even when the container experiences vibration during transportation. The foregoing effects permit the fragile object to be stored for a relatively long period of time without deformation in the liquid.

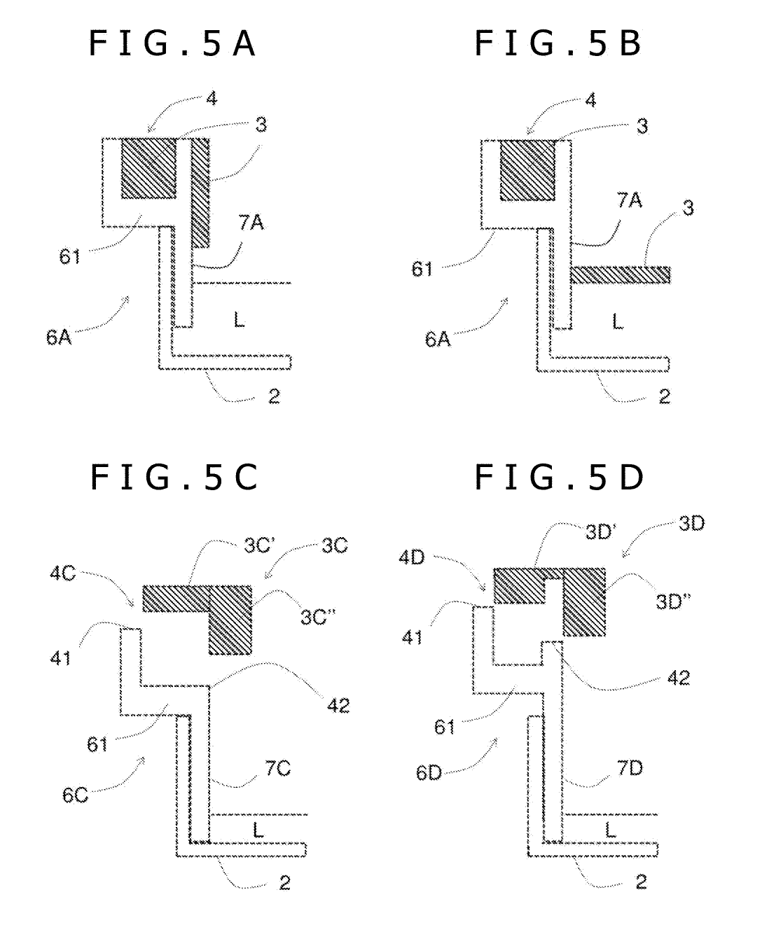

The member depicted in FIG. 4 may be modified as depicted in FIGS. 5A to 5D, which are schematic sectional views. Incidentally, the same components as those in the devices pertaining to the first and second embodiments and the member pertaining to the third embodiment are indicated by the identical symbols, with their explanation omitted.

### FIRST MODIFIED EXAMPLE

As depicted in FIG. 5A, a member 6A pertaining to the first modified example of the present disclosure is characterized in that the sealing medium 3 is fixed in the groove 4 and onto the inner side surface of the member 6A and that a fitting part 7A is not an annular groove but takes on an annular wall that protrudes downward from the bottom surface of a base 61 of the member 6A. The fitting part 7A is formed so as to have an outer side surface of the fitting part 7A in tight contact with the inner side surface of the container 2. The fitting part 7A is long enough for a lower end of the fitting part 7A to dip (or extend) into the liquid L held in the container 2. The sealing medium 3 melts on heating, and the sealing medium 3 in a molten state drips down along the inner side surface of the sealing medium 3 to reach the liquid surface and flow toward the center of the liquid surface. As the result, the sealing medium 3 has a circumferential edge of the sealing member 3 fixed to the inner side surface of the member 6A (FIG. 5B). This arrangement makes it relatively easy to remove the sealing medium 3 from the liquid surface by simply removing the member 6A from the container 2.

### SECOND MODIFIED EXAMPLE

It is depicted in FIG. 5C that a member 6C pertaining to the second modified example of the present disclosure does not have a sealing medium fixed to a groove 4C. According to this modified example, the groove 4C is not an annular groove but an annular wall that protrudes upward from the top of the base 61 of the member 6C. According to this modified example, the groove 4C is constructed such that the inner part (inner wall) 42 of the groove 4C is lower than the outer wall 41 of the groove 4C and is not protruding from the base 61. The member 6C pertaining to the present disclosure is put to use in such a way that the member 6C is fixed to the container (not depicted) and a sealing medium 3C in an annular shape is placed in the groove 4C and then heated. The sealing medium 3C to be employed in this modified example consists of an annular sealing medium 3C′ that can be placed in the groove 4C of the member 6C and an annular sealing medium 3C″ that can be placed on the inner side surface of the member 6C, which are integrally molded. The sealing medium 3C melts in the groove 4C and on the inner side surface of the member 6C, and drips down along the inner side surface of a fitting part 7C.

In this manner, the member 6 pertaining to the present disclosure may have the sealing medium 3 mounted later. Moreover, it is not necessary for the sealing medium 3 to be exactly adjusted in height with the inner part 42 and the outer wall 41 of the groove 4C, because the inner part 42 of the groove 4C does not protrude (or extend) from the base 61. The modified example mentioned herein is characterized in that the fitting part 7C is approximately as long as the height of the inner side surface of the container 2. This permits the fitting part 7C to completely cover the inner side surface of the container 2. Thus, the sealing medium 3 can be led to the liquid surface along the inner side surface of the member 6C even in the case where the liquid level in the container 2 is low, as depicted in FIG. 5C.

In the present modified example, the member 6C may be used in such a way that the member 6C is fixed to the container, the annular sealing member 3C is placed in the groove 4C, and the lid (not depicted) is placed on the groove 4C. When the sealing medium 3C melts in this state, the lower end of the lid sinks into the sealing medium 3C′ which is melting, and finally reaches the top of the groove 4C. When the lower end of the lid reaches the top of the groove 4C, that portion of the sealing medium 3C′ which is in the outside of the lid has nowhere to go and stays between the lid and the outer wall 41 of the groove 4C. The sealing medium is solidified in this state so that the sealing medium staying between the lid and the outer wall 41 of the groove 4C fixes the lid to the groove 4C.

According to the present modified example, the groove 4C is not an annular groove which permits the lid to enter the groove 4C. This structure may be applied to the grooves 4 and 4B depicted in FIGS. 3A to 3C. In this case, for example, the lid may take on a discoid shape, which has a smaller diameter than the outside diameter of the grooves 4, 4B, and 4C, which can obviate (or eliminate) the necessity of fitting the lid into the groove. In addition, with the lid having a discoid shape, it is only necessary to place the lid on the groove to achieve the desired object.

### THIRD MODIFIED EXAMPLE

In a member 6D pertaining to the third modified example of the member 6 of the present disclosure, as depicted in FIG. 5D, a sealing medium is not fixed in a groove 4D. A fitting part 7D protrudes downward from the lower surface of the base 61 of the member 6D and the fitting part 7D is longer than the height of the inner side surface of the container 2. This structure permits the fitting part 7D to entirely cover the inner side surface of the container 2. In this modified example, the groove 4D consists of two annular walls protruding upward from the upper surface of the base 61 of the member 6D. The two annular walls include an inner annular wall 42 and an outer annular wall 41 higher than the inner annular wall 42, and a sealing medium 3D is higher than the inner annular wall 42 and lower than the outer annular wall 41.

When the member 6D is put to use, the member 6D is fixed to the edge of the container 2 which holds a liquid and a fragile object, and the sealing medium 3D in an annular shape is placed on the groove 4D and then heated. This modified example employs the sealing medium 3D which consists of an annular sealing medium 3D′ and an annular sealing medium 3D″ which are integrally molded, with the annular sealing medium 3D′ being placeable on the groove 4D of the member 6D and the sealing medium 3D″ being placeable on the inner side surface of the member 6D. When the sealing medium 3D melts, the annular sealing medium 3D′ positioned above the inner annular wall 42 and the annular sealing medium 3D″ drip down along the inner side surface of the member 6D to reach the liquid surface. The lower portion of the sealing medium 3D′ stays (or remains) in the groove 4D, thereby properly fixing the lid fitting in the groove 4D.

As mentioned above, the sealing medium 3 may be formed arbitrarily in conformity with the shape of the member 6. For example, the sealing medium 3 in an annular shape may be replaced by one in a discoid shape. When the sealing medium 3 in a discoid shape melts, the outer part of the sealing medium 3 may drip on the radially outer side of the liquid surface, and at the same time the inner part of the sealing medium 3 may drip on the radially inner side of the liquid surface.

In addition, the fitting part 7D may be properly adjusted in length according to the height of the container and the height of the surface of the liquid in the container. The fitting part 7D is not specifically restricted in length. For example, the length of fitting part 7D may range from 16 to 25 mm, preferably from 16 to 22 mm, and more preferably from 16 to 19 mm, for example.

It is noted from the foregoing that the modified examples of the present disclosure employ the member 6A, 6C, and 6D which can properly cover the surface of the liquid in the container, thereby preventing air from entering the gap between the liquid surface and the sealing medium, with the result that the liquid in the container does not wave nor does the liquid come into contact with external air and the fragile object in the liquid does not move even when the container experiences vibration during transportation. The foregoing effects permit the fragile object to be stored for a relatively long period of time without deformation in the liquid.

The foregoing demonstrates the devices and members pertaining to the embodiments 1 to 3 of the present disclosure. They are not intended to restrict the scope of the present disclosure. They may be properly modified and the modified products may be properly combined together to design the device and member differing in shape and structure, by those who are skilled in the art. For example, it will be possible to design the device 1 and the member 6 differing in shape and structure by properly combining the structures such as the grooves 4 and 4B and the sealing medium 3 of the device 1, and the grooves 4C and 4D and the sealing mediums 3C and 3D of the member 6.

The components used in the present disclosure may be substituted by arbitrary components which can function in the same way or may be added with arbitrary structure.

### EXAMPLE

The present disclosure will be described below in more detail with reference to an example, which is not intended to restrict the scope of the disclosure.

### Example 1

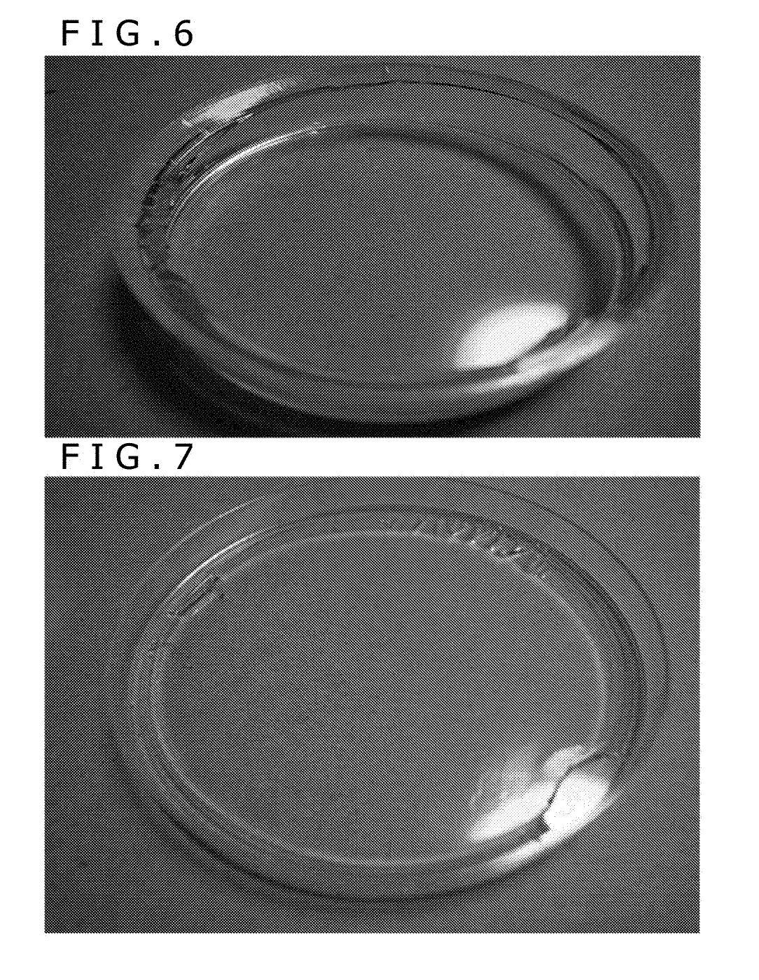

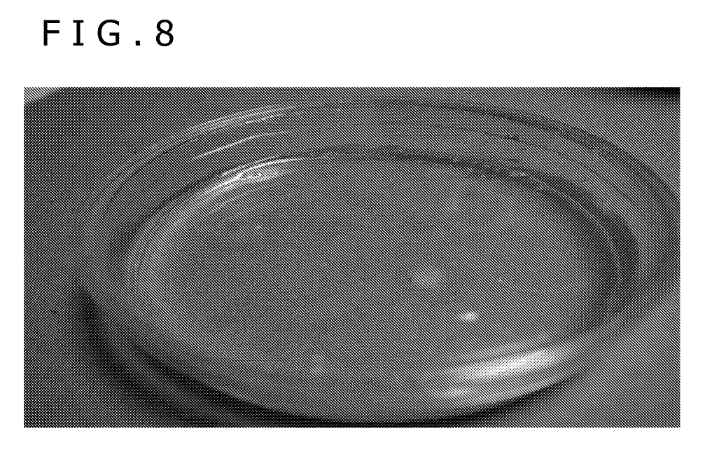

A gelatin in a molten state was applied to the inner side surface of a Petri dish (Nunc®), 10 cm in diameter, and the inner surface of a lid for the Petri dish, and then the dish and lid coated with gelatin were cooled in a refrigerator for one hour to bring about gelation. The gelled gelatin adhering to the inner side surface of the dish is shown in FIG. 6, and the gelled gelatin adhering to the inner surface of the lid is shown in FIG. 7. The dish was then charged with water and closed by the lid, and heated at approximately 40° C. until the gelatin melts. After the molten gelatin covered the water in the dish, the dish was refrigerated again for one hour to bring about gelation. It was found as shown in FIG. 8 that the gelled gelatin covered the water in the dish, thereby preventing the water in the dish from waving and moving.

The detailed description above describes a method that allows a fragile object to retain its form in a liquid, and also to a device and components of the device to put the method into practice. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A method for keeping a shape of a fragile material in a liquid in a container, the method comprising:
covering a surface of the liquid with a sealing material configured to melt and solidify according to a temperature change; and
solidifying the sealing material to cover the surface of the liquid.

2. The method according to claim 1, wherein the fragile material is a sheet-shaped cell culture of skeletal myoblasts.

3. The method according to claim 1, wherein the sealing material has a melting point of 0° C. to 45° C. and a solidifying point of 0° C. to 45° C.

4. The method according to claim 1, wherein the sealing material includes at least one selected from a group consisting of fatty ester, gelatin, polysaccharide, and polymeric compound.

5. The method according to claim 1, wherein the container includes a lid, and the method further comprising:
adhering the sealing material to the lid of the container in a solidified state.

6. The method according to claim 5, further comprising:
heating the solidified sealing material on the lid of the container to a molten sealing material, and wherein the molten sealing material adhering to the lid of the container drips from an inner surface of the lid in a radial direction onto to the surface of the liquid.

7. The method according to claim 1, wherein the container has an annular groove configured to receive a solidified sealing material, the annular groove having an inner wall and an outer wall, the method further comprising:
adhering solidified sealing material in the annular groove;
adhering solidified sealing material on an inner side surface of the container; and/or
adhering solidified sealing material to a lid of the container.

8. The method according to claim 7, further comprising:
heating the fragile material, the liquid, and the container to melt the solidified sealing material in the annular groove, on the inner side surface of the container, and the lid of the container;
dripping the molten sealing material from the annular groove, the inner side surface of the container and the lid of the container onto the surface of the liquid.

9. The method according to claim 8, wherein a height of the inner wall is lower than a height of the outer wall, the method further comprising:
dripping the sealing material down from the annular groove on the inner side surface of the container in a circumferential direction of the container.

10. A device for keeping a fragile material in a liquid, the device comprising:
a container configured to hold the fragile material; and
a sealing material configured to melt and solidify according to a temperature change.

11. The device according to claim 10, further comprising:
a lid for the container, and wherein the sealing material in a solidified state is configured to adhere to the container and/or the lid.

12. The device according to claim 10, wherein the container has an annular groove configured to receive the sealing material, the annular groove having an inner wall and an outer wall.

13. The device according to claim 12, wherein a height of the inner wall is lower than a height of the outer wall.

14. The device according to claim 10, wherein the fragile material is a sheet-shaped cell culture of skeletal myoblasts.

15. The device according to claim 10, wherein the sealing material has a melting point of 0° C. to 45° C. and a solidifying point of 0° C. to 45° C., and the sealing material includes at least one selected from a group consisting of fatty ester, gelatin, polysaccharide, and polymeric compound.

16. The device according to claim 10, wherein the container includes a lid, the lid having solidified sealing material fixed to a lower surface of the lid.

17. The device according to claim 10, wherein the container has an annular groove configured to receive a solidified sealing material, the annular groove having an inner wall and an outer wall.

18. The device according to claim 17, wherein a height of the inner wall is lower than a height of the outer wall.

19. An annular member capable of adhering to an edge of a container holding a flexible material, the annular member comprising:
an upper annular groove configured to receive a sealing material, the sealing material configured to melt and solidify according to a temperature change, the upper annular groove having an inner wall and an outer wall, and wherein a height of the inner wall is lower than a height of the outer wall.

20. The annular member according to claim 19, further comprising:
a lower annular groove configured to be received on the edge of the container.
